(19) United States
(12) Patent Application Publication
(10) Pub. No.: US 2019/0282722 A1
(43) Pub. Date: Sep. 19, 2019

(54) GAS PROCESSING APPARATUS

(71) Applicant: Kabushiki Kaisha Toshiba, Tokyo (JP)

(72) Inventors: Akio UI, Tokyo (JP); Masato AKITA, Kawasaki (JP); Yosuke SATO, Kawasaki (JP)

(73) Assignee: Kabushiki Kaisha Toshiba, Tokyo (JP)

(21) Appl. No.: 16/111,396

(22) Filed: Aug. 24, 2018

(30) Foreign Application Priority Data
Mar. 16, 2018 (JP) 2018-49899

(51) Int. Cl. A61L 9/22; B01D 53/32; B01D 53/66; B01D 53/82; B01D 53/86; B01D 53/75; B01D 53/34; B01D 53/04; A61L 9/014

(52) U.S. Cl. CPC A61L 9/22; A61L 2209/111; B01D 53/66; B01D 53/82; B01D 53/8675; B01D 53/75; B01D 53/346; B01D 53/04; A61L 9/014; B01D 2253/102; B01D 2257/406; B01D 2257/404; B01D 2257/90; B01D 2257/91; B01D 2257/106; B01D 2259/4508; B01D 2255/20; A61L 2209/14; A61L 2209/212; B01D 53/32

(57) ABSTRACT

A gas processing apparatus of an embodiment includes a gas processing unit, a flow forming unit, an AC power supply, and first and second filters. The gas processing unit includes a plurality of stacks each having a dielectric substrate, a first to a third electrode. The flow forming unit forms a flow of a target gas flowing toward the gas processing unit. The AC power supply applies an AC voltage across the first, second electrodes and the third electrode so as to generate plasma induced flows of the target gas between the dielectric substrates. The first filter is disposed at an upstream of the gas processing unit, and removes ozone. The second filter is disposed at a downstream of the gas processing unit, and removes ozone.

### CROSS REFERENCE TO RELATED APPLICATION

This application is based upon and claims the benefit of priority from Japanese Patent Application No. 2018-049899, filed on Mar. 16, 2018; the entire contents of which are incorporated herein by reference.

### FIELD

Embodiments described herein relate generally to a gas processing apparatus which decomposes gas by using electric discharge.

### BACKGROUND

Atmospheric gas in a living space, a refrigerator, a warehouse, or the like or exhaust gas from a processing apparatus may contain toxic substances, malodorous substances, and the like. Small, high-efficiency gas decomposition apparatuses (including air purifiers, air purifying air-conditioners, and gas purifiers) are demanded for performing decomposition, sterilization, and the like (hereinafter referred to as gas decomposition) of such toxic substances, malodorous substances, and the like.

A gas decomposition apparatus of plasma actuator system (PA) decomposes gas by using plasma generated through electric discharge and a flow of ions. Specifically, a target gas is drawn in plasma through a flow of ions, and the target gas is oxidized and decomposed by OH radicals in the plasma. As a result of this, malodorous molecules and bacteria in the target gas are processed, and it becomes possible to perform deodorization and sterilization at high speed. However, the plasma also contains ozone (O₃) which is harmful to the human body, and the ozone flows out of a gas processing apparatus.

### BRIEF DESCRIPTION OF THE DRAWINGS

FIG. 1 is a side view illustrating an entire configuration of a gas decomposition apparatus 10 according to an embodiment.

FIG. 2 is an enlarged schematic view illustrating details of gas decomposition elements 20 constituting a processing unit U.

FIG. 3 is a graph representing a relation between an air velocity and a gas processing speed.

FIG. 4 is a graph representing a relation between an air velocity and a leakage ozone amount.

FIG. 5 is a graph representing a temporal change in a leakage ozone concentration.

### DETAILED DESCRIPTION

A gas processing apparatus of an embodiment includes a gas processing unit, a flow forming unit, an AC power supply, and first and second filters. The gas processing unit includes a plurality of stacks being away from each other and in parallel. Each stack includes a dielectric substrate and a first to a third electrode. The dielectric substrate has a first and a second main surface. The first and second electrodes are respectively disposed on the first and second main surfaces. The third electrode is disposed inside the dielectric substrate. The flow forming unit forms a flow of a target gas flowing toward the gas processing unit. The AC power supply applies an AC voltage across the first, second electrodes and the third electrode so as to generate plasma induced flows of the target gas between the dielectric substrates. The first filter is disposed at an upstream of the gas processing unit and removes ozone. The second filter is disposed at a downstream of the gas processing unit and removes ozone.

Hereinafter, embodiments will be described in detail while referring to the drawings.

### First Embodiment

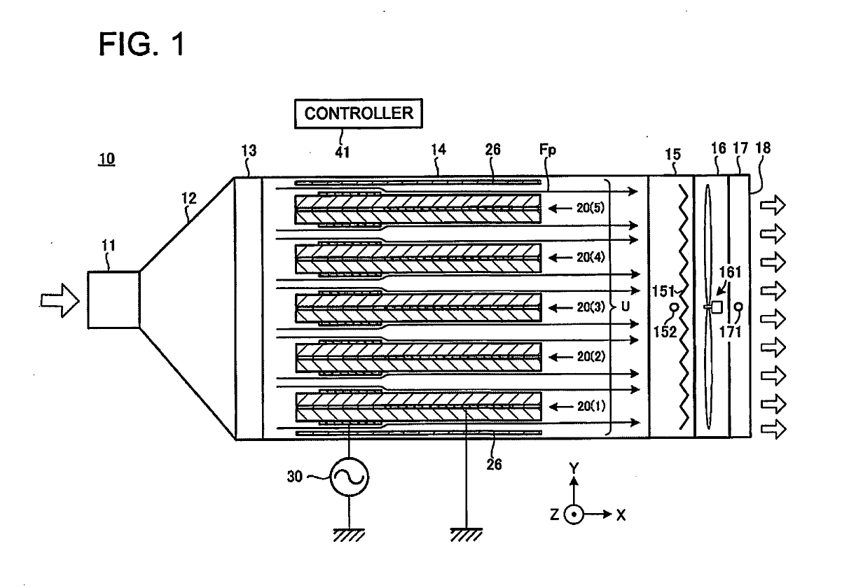

FIG. 1 illustrates an entire configuration of a gas decomposition apparatus 10 according to a first embodiment. The gas decomposition apparatus 10 decomposes a decomposition target (malodorous molecules, bacteria, virus) contained in a gas to be processed (atmosphere or process exhaust gas) by electric discharge generated by a high AC voltage applied across discharge electrodes and ground electrodes.

The gas decomposition apparatus 10 has a gas introduction port 11, a flow path expansion part 12, an upstream side filter 13, a gas decomposition chamber 14, a downstream side filter 15, a blowing unit 16, and a gas detector 17, and insides thereof are gas flow spaces.

A gas to be processed containing a decomposition target flows into the gas decomposition apparatus 10 from the gas introduction port 11. The gas decomposition chamber 14 and the blowing unit 16 contribute to this inflow. As will be described later, a plasma induced flow Fp generated in the gas decomposition chamber 14 and blow of air performed by the blowing unit 16 are combined to form a moderate flow of the gas to be processed, which makes it easy to perform efficient oxidation and decomposition in the gas decomposition chamber 14. The flow path expansion part 12 expands the flow path from the gas introduction port 11 to the upstream side filter 13 and the gas decomposition chamber 14.

The upstream side filter 13 and the downstream side filter 15 remove ozone in the gas to be processed. Note that details thereof will be described later.

In the gas decomposition chamber 14, a processing unit U including a plurality of gas decomposition elements 20(1) to 20(5) is disposed, so as to process the gas to be processed. The processing unit U functions as a gas processing unit having a plurality of stacks.

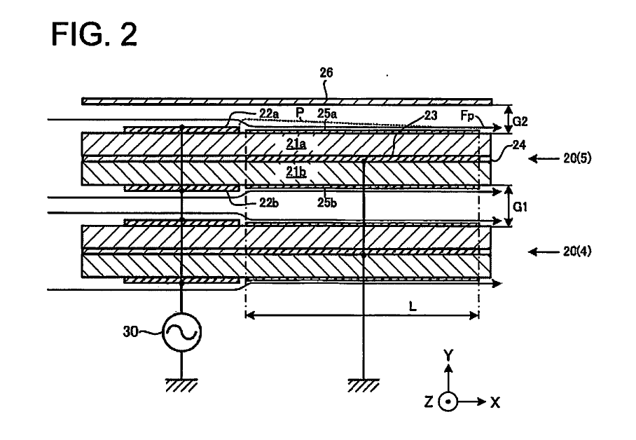

FIG. 2 illustrates, in an enlarged manner, details of gas decomposition elements (DBD-system plasma actuator) 20 constituting the processing unit U. The processing unit U has gas decomposition elements 20 (20(1) to 20(5)), and gas flow partitions 26. Here, the number of gas decomposition elements 20 included in the processing unit U is set to five, but, it can be changed appropriately and can be set to ten, for example.

The gas decomposition elements 20 have dielectric substrates 21 (21a, 21b), discharge electrodes 22 (22a, 22b), ground electrodes 23, insulating sealing layers 24, photocatalyst layers 25 (25a, 25b), and gas flow partitions 26.

The dielectric substrates 21a, 21b, the discharge electrodes 22a, 22b (first, second electrodes), the ground electrode 23 (third electrode), the insulating sealing layer 24, and the photocatalyst layers 25a, 25b of one gas decomposition element 20 function as a stack.

The dielectric substrates 21 are substrates of dielectric material (for example, quartz, silicon rubber, or polyimide). For example, quartz plates with a thickness of 1 mm can be used as the dielectric substrates 21. The discharge electrodes 22 and the ground electrode 23 are constituted of a conductor of metal or the like. For example, a gold (Au) thin film can be formed on the dielectric substrates 21 by means of sputtering or plating, so as to make the discharge electrodes 22 and the ground electrode 23.

Regarding the discharge electrodes 22 (22a, 22b) and the ground electrode 23, a size of the former is 5 mm×150 mm, and a size of the latter is 10 mm×150 mm, for example, in a flow direction of the gas to be processed (X direction) and a depth direction (Z direction). Specifically, a length L in the X direction of the former is shorter than that of the latter. As will be described later, plasma P and a plasma induced flow Fp are generated in a range of the length L on the discharge electrodes 22 side, so as to perform oxidation and decomposition processing. The discharge electrodes 22 (22a, 22b) and the ground electrode 23 are disposed by being displaced in the X direction (in a flow direction of the plasma induced flow Fp to be described later).

The dielectric substrates 21 of adjacent gas decomposition elements 20 are disposed to have a gap G1 of not less than 2 mm nor more than 6 mm (2 mm, for example). The dielectric substrates 21 of the top (and bottom) gas decomposition element 20 are disposed with a gap G2 of not less than 1 mm nor more than 3 mm (2 mm, for example), from the gas flow partition 26. Note that in this case, the thicknesses of the discharge electrodes 22 and the thicknesses of the photocatalyst layers 25 can be ignored with respect to the gaps G1, G2. By setting the gaps G1, G2 as described above, it is possible to effectively utilize oxygen radials, particularly OH radicals in the plasma P, to thereby efficiently decompose the decomposition target.

The insulating sealing layer 24 is a dielectric film for suppressing reverse discharge near the ground electrode 23. For example, a glass film, a silicon oxide film, or a silicone agent can be used as the insulating sealing layer 24. The insulating sealing layer 24 is provided for preventing a hindrance of the oxidation and decomposition processing in the plasma P which is caused when the gas to be processed is brought into contact with the ground electrode 23 to generate unnecessary electric discharge.

The photocatalyst layers 25a, 25b are layers of photocatalyst material (TiO₂, for example), and are disposed in the vicinity of the plasma P or in the plasma P on the dielectric substrates 21. The photocatalyst layers 25a, 25b can be formed by, for example, applying a photocatalyst material.

The photocatalyst layers 25a, 25b are activated by light emission from the plasma P, and remove NOx or the like contained in the plasma P. Specifically, it becomes possible to remove the target more efficiently by the plasma P and the photocatalyst operating together.

Note that the gas decomposition elements 20 need not have the photocatalyst layers 25. However, when the gas decomposition elements 20 have the photocatalyst layers 25, it becomes possible to remove the target more efficiently.

A high-voltage AC power supply 30 applies a high AC voltage (a sinusoidal voltage with a frequency of 5 kHz to 70 kHz (10 kHz, for example) and a voltage (amplitude) of 3 kV to 10 kV (5 kV, for example) between the discharge electrodes 22a, 22b and the ground electrode 23. At this time, the inside of the gas decomposition apparatus 10 has a substantially atmospheric pressure.

By the high AC voltage from the high-voltage AC power supply 30, the plasma P of dielectric barrier discharge is generated in a region corresponding to the ground electrode 23 (a range corresponding to the length L) of an upper surface of the dielectric substrate 21a and a lower surface of the dielectric substrate 21b (on the discharge electrode 22 side). The plasma P includes positive ions and electrons. Due to a difference in the mass and electric properties of positive ions and electrons, a surface of the dielectric substrate 21 which is brought into contact with the plasma P is negatively charged in an AC cycle, resulting in that self-bias occurs in the plasma P. By the self-bias at a place on the dielectric substrate 21 on which only the ground electrode 23 is disposed (the discharge electrode 22 is not disposed), positive ions move in an X-axis positive direction, collide with molecules of the gas to be processed, and advance the gas. As a result of this, a flow containing both of ions and gas (plasma induced flow Fp) in an X-axis positive direction is generated. This plasma induced flow Fp draws the gas to be processed in the plasma P, to thereby contribute to efficient processing of gas (efficient oxidation and decomposition of the target).

The plasma P contains oxygen radicals (O, O., O₂., OH, HO₂) generated through electric discharge of the gas to be processed containing vapor (atmosphere), and ozone (O₃) (refer to an equation (1)).

O₂+electric discharge→O,O.,O₂.,OH,HO₂,O₃ (1)

The oxygen radicals are, for example, 0 (oxygen atom radicals), O. (excited oxygen atom radicals), O₂. (excited oxygen molecule radicals), OH (OH radicals), and HO₂ (hydroperoxy radicals). The oxygen radicals have strong oxidizing power, and strongly oxidize and decompose (deodorize and sterilize) the decomposition target (malodorous molecules, bacteria, virus) contained in the gas to be processed. Among the oxygen radicals, OH radicals have particularly strong oxidizing power (deodorization and sterilization operations). For this reason, OH radicals in the plasma P particularly contribute to the deodorization and sterilization.

However, OH radicals have strong reactivity, and thus they easily react with O₂ molecules and N₂ molecules to be deactivated (refer to equations (2), (3), (4)).

OH+O₂→HO₂+O (2)

OH+N₂→NO+NH (3)

OH+N₂→NO₂+N (4)

A life span of OH radicals in the atmospheric pressure is 1 ms or less, and when a flow velocity of gas is set to 1 m/s, for example, a moving distance at this time becomes 1 mm. Specifically, OH radicals exist only in a range within 1 mm from the plasma P (substantially in the plasma P), and there is no chance that OH radicals directly exert influence on the human body and the like.

The oxidation and decomposition of the target are caused by a certain amount of OH radicals which are continuously generated in the plasma P. This decomposition reaction can be regarded as a first-order reaction with respect to decomposition target molecules. Accordingly, a concentration C of the decomposition target which flows out of the processing unit U can be represented by an equation (5).

C=C0·exp (−kτ) (5)

C0: Concentration of decomposition target which flows into processing unit U

k: Rate constant (k=vt*Ct)

vt: Reaction rate between molecules of decomposition target and OH radicals

Ct: Concentration of OH radicals (depending on input power per unit length)

τ: Time (second) of passing region (length L) where OH radicals exist

Since ozone (O₃) generated in the plasma P has a long life span (several tens of minutes at room temperature), it flows out of the gas decomposition apparatus 10 (processing unit U) to be diffused in the room. As will be described later, the gas decomposition apparatus 10 is designed to prevent a concentration of ozone to be flowed out from exceeding an allowable concentration. Note that the allowable concentration of ozone is 0.1 ppm (Japan Society for Occupational Health).

The blowing unit 16 is, for example, a blower or an exhauster, and in this case, a fan 161 which is driven by a motor is used. The blowing unit 16 functions as a flow forming unit which forms a flow of a target gas which flows toward the processing unit U (the gas processing unit).

Here, the blowing unit 16 is disposed at a downstream of the gas decomposition chamber 14, and sucks out the target gas from the gas decomposition chamber 14. However, it is also possible that the blowing unit 16 is disposed at an upstream of the gas decomposition chamber 14 to push out the target gas to the gas decomposition chamber 14. When the blowing unit 16 is disposed at the downstream of the gas decomposition chamber 14, the blowing unit 16 is preferably disposed at a position further on the downstream side relative to the downstream side filter 15. This is because the downstream side filter 15 is preferably in close vicinity to the processing unit U, as will be described later.

As already described above, since the gas to be processed is drawn in by the plasma induced flow Fp in the processing unit U, it is possible to make the gas to be processed to be flowed into or flowed out of the gas decomposition apparatus 10 even if the blowing unit 16 is not provided. However, by increasing a flow rate of the gas to be processed with the use of the blowing unit 16, it is possible to further increase a gas processing amount. Specifically, a flow velocity v of the target gas takes a value as a result of adding vp of the plasma induced flow Fp itself and a flow velocity of the target gas ascribable to the blowing unit 16, as represented by an equation (6).

v=vp+vf (6)

A gas processing amount in a unit time is in proportion to a flow rate (flow velocity v), so that a total processing amount increases with an increase in the flow velocity. However, when the flow velocity v of the target gas is increased, the amount of the decomposition target in the gas to be processed which flows out of the gas decomposition apparatus 10 is also increased over the amount which is possibly decomposed by the amount of OH radicals generated in the plasma P and finally the total processing amount decreased. For this reason, it is preferable to increase the flow rate (flow velocity) in the blowing unit 16 within a proper range keeping the large processing amount.

The gas detector 17 is a section where a gas to be discharged from the gas decomposition apparatus 10 is detected, and an ozone sensor 171 is disposed. The ozone sensor 171 detects an ozone concentration in the gas to be discharged from the gas decomposition apparatus 10. As will be described later, a blowing amount in the blowing unit 16 is controlled based on the detected ozone concentration. The gas after being passed through the gas detector 17 flows out of a gas outlet 18.

A controller 41 is configured by a combination of hardware (CPU: central processing unit) and software (program), for example, and controls the gas decomposition apparatus 10. Note that the controller 41 may also be configured by only the hardware. The controller 41 can be used for performing controls of B to F to be described later (heating control of filter, control of flow rate, control for preventing back-flow of ozone, switching control of operation state, and control of cleaning in room using ozone).

### (Control of Leakage Ozone Concentration)

Hereinafter, details of control of leakage (flowed-out) ozone concentration will be described.

A. Absorption by Filters

The upstream side filter 13 and the downstream side filter 15 are ozone removal filters that remove ozone from the gas to be processed. Hereinafter, details thereof will be described. The ozone removal filter is a filter that decomposes ozone, and has a metal catalyst (M: an oxide of Mn, CO, Ni, for example, manganese dioxide: MnO₂, for example), or activated carbon. The metal catalyst operates as a catalyst to accelerate the decomposition of ozone (refer to equations (7) to (9)). The activated carbon reacts with ozone to be CO₂ (refer to an equation (10)).

2O₃+M→3O₂+M (7)

O₃+M→MO+O₂ (8)

O₃+MO→M+2O₂ (9)

2O₃+3C→3CO₂ (10)

The metal catalyst has a high ozone processing capability. Further, the metal catalyst has a self-cleaning function, and a life span thereof is long (a metal state and an oxide state are switched due to a reaction with ozone as represented in the equations (8), (9), or the metal state is maintained before and after the processing of ozone as represented in the equation (7)). However, the metal catalyst is not adequate to processing of ammonia, nitric acid, and the like (a surface of the catalyst is poisoned, and the catalyst activity is lowered). Further, temperature dependence is large, so that when a temperature is low at the beginning of the operation, the ozone processing performance is not good. On the other hand, the activated carbon can also adsorb and remove ammonia, nitric acid, and the like, but, the processing capability thereof is lowered due to the adsorption. Further, the activated carbon is burnt and consumed when it reacts with ozone, so that a life span thereof is relatively short.

In this case, the upstream side filter 13 uses the activated carbon, and the downstream side filter 15 uses the metal catalyst. By processing a relatively small amount of ozone, ammonia, nitric acid, and the like with the use of the upstream side filter 13, and processing a large amount of ozone with the use of the downstream side filter 15, it becomes easy to perform stable processing of ozone.

Basically, the gas to be processed flows from the upstream side filter 13 toward the processing unit U, so that there is a small possibility that ozone generated in the processing unit U flows backward to the upstream side filter 13. However, there is a possibility that a part of high-concentration ozone generated in the processing unit U diffuses in a direction opposite to the flow of the gas to be processed, and flows backward as a relatively small amount of ozone. This back-flow is easily generated particularly right after the start of operation in which the flow of the gas to be processed (a blowing state in the whole gas decomposition apparatus 10) is unstable.

The upstream side filter 13 functions effectively for preventing such a back-flow of ozone. The activated carbon is suitable for processing this relatively low amount of ozone. The temperature is relatively low right after the start of the operation, so that the activated carbon is more suitable than the metal catalyst which has the large temperature dependence.

A large amount of ozone generated in the processing unit U constantly flows into the downstream side filter 15, so that the metal catalyst is preferably used for the downstream side filter 15. By processing ozone with the use of the downstream side filter 15 while processing ammonia, nitric acid, and the like with the use of the upstream side filter 13, it is possible to process a large amount of ozone while preventing the metal catalyst from being poisoned.

As already described above, the activated carbon has a relatively short life span. For this reason, it is preferable to design such that the activated carbon of the upstream side filter 13 is easily replaceable. For example, the upstream side filter 13 is prepared as a cassette which can be easily removed from the gas decomposition apparatus 10, and the activated carbon is replaced together with the cassette. Note that it is also possible to design such that the downstream side filter 15 is also easily replaceable in addition to the upstream side filter 13.

As described above, by appropriately selecting the material for ozone removal used for the upstream side filter 13 and the downstream side filter 15, it becomes possible to perform efficient ozone removal.

B. Heating Control of Filter

As already described above, the metal catalyst has the large temperature dependence. For this reason, the downstream side filter 15 includes not only the catalyst but also a heater 151, and a temperature sensor 152. The heater 151 increases a temperature of the catalyst, particularly the metal catalyst, to make it possible to perform efficient processing. As the heater 151, for example, a resistive electric heater can be used. The temperature sensor 152 is used for monitoring a temperature of the downstream side filter 15 (catalyst) to maintain an optimum temperature.

Specifically, by heating the downstream side filter 15 by using the heater 151, it becomes easy to efficiently decompose ozone with the use of the downstream side filter 15. Further, it is possible to monitor the temperature of the downstream side filter 15, control a heating state created by the heater 151 based on the temperature, and maintain the temperature of the downstream side filter 15 to fall within a proper range (50 to 80° C., for example, 60° C.).

Here, if the processing unit U is operated after the temperature of the downstream side filter 15 (metal catalyst) is raised to a certain degree, it is possible to securely remove ozone generated in the processing unit U, with the use of the downstream side filter 15. For example, the controller 41 controls the gas decomposition apparatus 10, and when the temperature of the downstream side filter 15 exceeds a threshold value (50° C., for example), the driving of the processing unit U is started (the AC voltage is applied).

C. Control of Flow Rate

The ozone sensor 171 detects the leakage ozone concentration, and based on this detection result, the flow rate in the blowing unit 16 is controlled. For example, when the detected ozone concentration exceeds a threshold value, the flow rate in the blowing unit 16 is decreased. When it is designed as above, a proportion of ozone with respect to the discharged gas to be processed is reduced, resulting in that the ozone concentration is lowered.

D. Control for Preventing Back-Flow of Ozone

As already described above, there is a possibility that a part of ozone flows backward at a time right after the start of the operation and the like. In order to avoid this, it is possible to consider that the blowing unit 16 is operated before the operation of the processing unit U. By operating the processing unit U after the blowing unit 16 is operated and the blowing state in the whole gas decomposition apparatus 10 is stabilized, it is possible to prevent ozone generated in the processing unit U from flowing backward to the upstream side. Specifically, it is only required to sequentially operate the blowing unit 16 and the processing unit U by leaving time (about 10 seconds to 120 seconds, for example, 30 seconds) in between. This sequential operation can be controlled by the controller 41. However, it is also possible to use a simple timer.

E. Switching Control of Operation State

As will be indicated in Example 3 to be described later, in the gas decomposition apparatus 10 (the processing unit U), there was a tendency that ozone is easily discharged relatively at the beginning of the operation (up to about 5 minutes from the start of the operation, for example).

For this reason, by switching the operation state from a low operation state to a normal operation state in accordance with transition of operation, it becomes possible to perform gas processing with no leakage of ozone. For example, the controller 41 performs control so that the processing unit U (the gas processing unit) or the blowing unit 16 (the flow forming unit) operates in a first mode (low operation mode), and then the processing unit U (the gas processing unit) or the blowing unit 16 operates in a second mode (normal operation mode). When the low operation state continues for a predetermined time (5 minutes or more, for example), the operation state is switched from the low operation state to the normal operation state.

By changing power of at least either of the processing unit U and the blowing unit 16, the level of the operation state can be changed. For example, it is possible to switch the normal operation and the low operation in accordance with the magnitude of the voltage which is applied to the processing unit U. Further, it is possible to switch the normal operation and the low operation in accordance with the magnitude of an air velocity (air flow rate) of gas provided by the blowing unit 16. It is also possible to switch the normal operation and the low operation by changing powers of both of the processing unit U and the blowing unit 16.

F. Control of Cleaning in Room Using Ozone

In the above description, it is an object to protect health of human being by suppressing the concentration of leakage ozone from the gas decomposition apparatus 10. On the contrary, it is also possible to consider to positively utilize ozone generated in the processing unit U. Specifically, it is possible to moderately deodorize the inside of room by discharging ozone with relatively low concentration (of about 0.1 ppm, for example) from the gas decomposition apparatus 10.

In order to secure a moderate ozone concentration, for example, it is only required to make powers of both of the processing unit U and the blowing unit 16 to be larger than normal powers. By increasing the voltage which is applied to the processing unit U, it is possible to increase a generation amount of ozone generated in the processing unit U. When the powers (the flow rate and the flow velocity) of the blowing unit 16 are increased, even if the upstream side filter 13 and the downstream side filter 15 are provided, an amount of ozone to be decomposed by these filters becomes small. As a result of this, it is possible to discharge ozone with a moderate concentration from the gas decomposition apparatus 10.

For example, the controller 41 switches a first state where the processing unit U (the gas processing unit) or the blowing unit 16 (the flow forming unit) operates in a third mode (normal operation mode) to discharge ozone with a first concentration (low concentration) and a second state where the processing unit U or the blowing unit 16 operates in a fourth mode (high operation mode) to discharge ozone with a second concentration (high concentration). This third mode (normal operation mode) can be set to the same as the second mode (normal operation mode) described in “E. switching control of operation state”.

In this case, it is possible to control the concentration of ozone in the normal operation mode and the high operation mode by using the ozone sensor 171. For example, in response to the magnitude of the ozone concentration, the ozone sensor 171 adjusts the powers of the processing unit U and/or the blowing unit 16.

The operation in the high operation mode is preferably performed in a period of time when there is no person (in the middle of the night, for example). For instance, the time of the start and the time of the termination of the operation in the high operation mode are set, and based on this setting, the controller 41 controls the gas decomposition apparatus 10. The atmosphere containing ozone with a moderate concentration which is discharged in the room is ventilated according to a standard (0.5 times or more/h) based on the building law in Japan, and thus the atmosphere is used for the gas processing (deodorization, sterilization) and then gradually exhausted.

### EXAMPLES

Hereinafter, examples will be described.

(1) Example 1

Ten gas decomposition elements 20 were stacked with a gap of 2 mm provided therebetween to be formed as the processing unit U. Here, regarding the discharge electrodes 22 (22a, 22b) and the ground electrode 23, a size of the former was set to 5 mm×150 mm, and a size of the latter was set to 10 mm×150 mm in a length direction (X direction) and a depth direction (Z direction).

One sheet of activated carbon with a thickness of 20 mm was used for the upstream side filter 13, and two sheets of MnO₂ catalyst with a thickness of 10 mm were used for the downstream side filter 15.

The blowing unit 16 was brought into operation, and the flow velocity vf in the gap G1 in the processing unit U was set to 0.4 [m/s].

After that, an AC voltage with 10 kHz and an amplitude of 5 kV was applied between the discharge electrodes 22 and the ground electrode 23. As a result of this, plasma of dielectric barrier discharge was generated from an end portion of the discharge electrode 22 toward a position of 10 mm above a surface of the dielectric substrate 21 on the discharge electrode 22 side, to thereby generate the plasma induced flow Fp. At this time, the flow velocity vp of the plasma induced flow Fp itself was about 0.3 [m/s] (including a pressure loss when disposing the catalyst, specifically, in the upstream side filter 13 and the downstream side filter 15). From the equation (6), the flow velocity v (=vp+vf) of the gas to be processed becomes 0.7 [m/s].

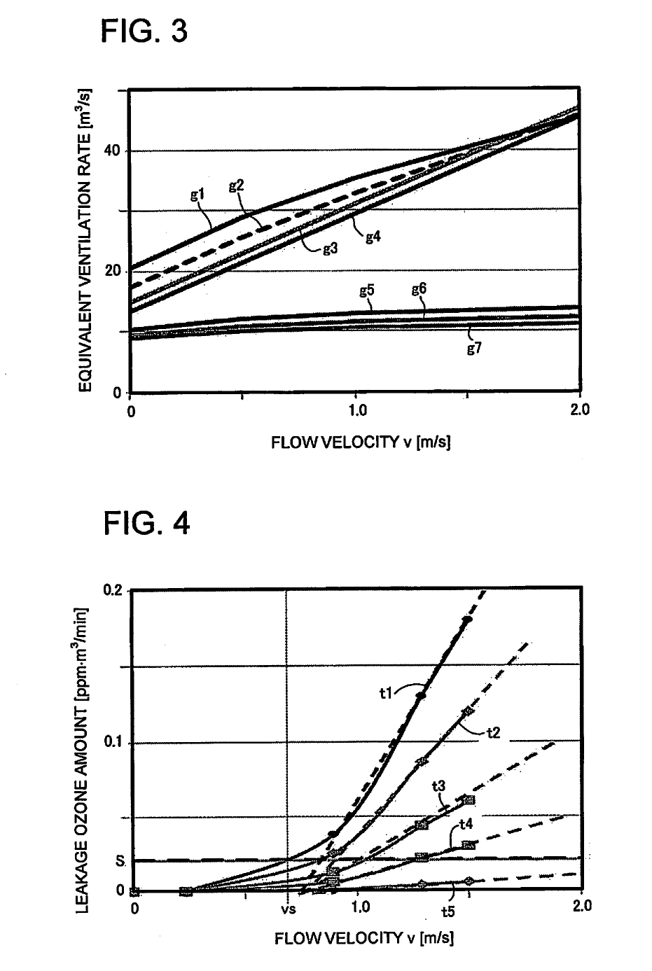

FIG. 3 is a graph representing a relation between an air velocity and a gas processing speed. Here, analysis was conducted by using OH radical concentrations determined through an experiment. A horizontal axis in FIG. 3 indicates the flow velocity v of the gas to be processed, and a vertical axis in FIG. 3 indicates processing capability (processing rate) of the decomposition target in terms of reduced ventilation rate [m³/s] (a ventilation rate of atmosphere indicating equivalent performance).

Graphs g1 to g7 correspond to acetaldehyde (CH₃CHO), the entire tobacco odor (tobacco odor in which ammonia, acetic acid, and acetaldehyde being main components are integrated in a ratio of 1:1:2), acetic acid (CH₃COOH), ammonia (NH₃), ethylene, toluene, and hydrogen sulfide (H₂S) being decomposition targets, respectively.

The activated carbon of the upstream side filter 13 has a large power to adsorb and remove the decomposition targets corresponding to the graphs g1 to g4 (acetic acid and ammonia), and has a relatively small power to adsorb and remove the decomposition targets corresponding to the graphs g5 to g7 (ethylene and the like). For this reason, in the graph g1 to g4, the processing amount increases substantially in proportion to the flow velocity v. On the other hand, although the processing amount increases with the flow velocity v also in the graph g5 to g7, gradients of the graphs g5 to g7 are far smaller than those of the graphs g1 to g4.

FIG. 4 is a graph representing a relation between an air velocity and a leakage ozone amount, and indicating experimental results of ozone removal performed by two MnO₂ filters of the downstream side filter 15 in Example 1. A horizontal axis in FIG. 4 indicates the flow velocity v of the gas to be processed, and a vertical axis in FIG. 4 indicates an amount (discharge amount) of leakage ozone in a unit of [ppm·m³/min]. Graphs t1 to t5 correspond to temperatures of 22° C., 30° C., 40° C., 50° C., and 70° C., respectively.

When the gas to be processed containing ozone is brought into contact with MnO₂ when the gas passes through the downstream side filter 15, the ozone is oxidized and decomposed. As indicated by a dotted line in FIG. 4, an ozone removing capability is in proportion to a residence time (time of passing the length L) (inversely proportional to the flow velocity). Specifically, when the flow velocity is increased, the ozone removing capability is lowered.

Now, if eight-tatami room (space of about 30 m³) is supposed, a reduced rate ventilation rate stipulated in the building standard law in Japan (0.5 or more [times/h]) is 0.25 or more [m³/min]. At this time, based on the allowable concentration of ozone of 0.1 ppm, a safety standard value S (an upper limit ozone generation amount at which an ozone concentration in the room does not exceed the allowable concentration) becomes 0.025 [ppm·m³/min].

Specifically, the maximum flow velocity when the flow rate in the blowing unit 16 is adjusted within a range which does not exceed the safety standard value S, indicates the maximum gas processing capability. In the example of 22° C. in FIG. 4, “standard flow velocity vs=0.7 [m/s]” at an intersection point between the safety standard value S and the graph t1 corresponding to the temperature of 22° C., is preferable. Specifically, by setting the flow velocity v of the gas to be processed to equal to or less than the standard flow velocity vs, even if the ozone concentration in the room (here, 30-tatami room is supposed) is increased with the time, it is possible to secure 0.1 ppm or less. As already described above, in Example 1, it is designed such that the flow velocity v of the gas to be processed is set to 0.7 [m/s], to thereby maximize the processing amount of gas while suppressing the ozone concentration to fall within the allowable limit.

During a period of time in which ozone in the gas to be processed passes through the filter, the ozone reaches a hole wall surface in a hole structure of the filter, and is reacted to be removed. Accordingly, the ozone removing capability in the filter is in proportion to a filter passing time T (=thickness T of filter/flow velocity v). An amount Q [ppm·m³/min] of ozone which is passed without being removed by the downstream side filter 15 can be represented by an equation (11) (the equation (11) is effective in a range of “v0>U”).

Q=A·v·60·C0·(1−U/v0) (11)

A: Area of flow path of filter 15 [m²]

C0: Ozone concentration at front surface of filter [ppm]

U: Ozone removal ratio of downstream side filter 15 when flow velocity v of gas is 1 [m/s]

v0: Non-dimensional value of flow velocity v (v0=v [m/s]/1 [m/s])

Here, the ozone amount Q is set to an amount which does not exceed the aforementioned safety standard value S (0.025 [ppm·m³/min] in space of 30-tatami room) (Q≤S). When the flow velocity v is maximized in this range, it is safe and the decomposition efficiency of ozone becomes maximum. This is because the ozone passing amount represented by the equation (11) increases with the flow velocity v.

### (2) Example 2

As represented in an equation (12), ozone is generated through a triple collision reaction in heat generation. For this reason, as the temperature becomes high, ozone is decomposed more through an inverse reaction.

O+O₂+X→O₃+X+Q (12)

As illustrated in FIG. 4, the higher the temperature, the higher the ozone removing capability in the downstream side filter 15. The temperature of 50° C. or more is particularly effective, and at a temperature of 70° C. or more, there is no limit in terms of flow rate.

At this time, it is preferable that the downstream side filter 15 is set to be in close vicinity to the processing unit U. Because of heat from the processing unit U that is subjected to the electric discharge, the temperature of the downstream side filter 15 increases with the time, and maintains about 50° C.

The ground electrode 23 is disposed on the downstream side of the processing unit U. For this reason, it is also possible to make the downstream side filter 15 to be brought into contact with the processing unit U (the ground electrode 23). However, if the downstream side filter 15 is in close vicinity to and brought into contact with the ground electrode 23, there is a possibility that the plasma P becomes unstable. Accordingly, when the stability of the plasma P is taken into consideration, it is preferable that the downstream side filter 15 is separated by about 1 to 5 mm from a downstream end of the electrode 23. At this time, the downstream side filter 15 may be brought into contact with the dielectric substrate 21 and the insulating sealing layer 24 in the processing unit U.

### (3) Example 3

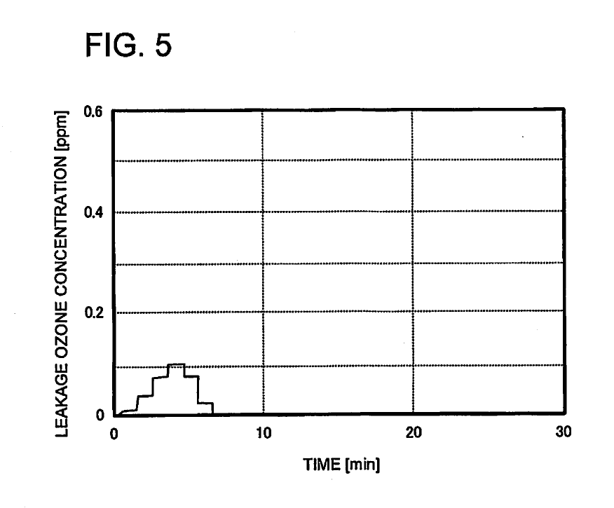

FIG. 5 is a graph representing a temporal change in an ozone concentration in the gas which flows out of the gas decomposition apparatus 10. A horizontal axis in FIG. 5 indicates a lapsed time from the start of operation of the gas decomposition apparatus 10, and a vertical axis in FIG. 5 indicates the ozone concentration. As illustrated in FIG. 5, regarding the ozone concentration in the gas to be flowed out, the ozone is slightly leaked at the beginning (about five minutes) of the start of the operation of the gas decomposition apparatus 10, and after that, it virtually stops leaking. This can be considered to correspond to the temperature change in the catalyst, particularly a surface of the catalyst in the filter 15. The temperature of the catalyst is low right after the start of the operation, and the leakage of ozone occurs. After that, by the processing of ozone, the temperature of the catalyst becomes high, and the leakage of ozone stops. Accordingly, as already described above, by switching the operation state from the low operation state to the normal operation state in accordance with the transition of operation, it becomes possible to perform gas processing with no leakage of ozone.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A gas processing apparatus, comprising:
a gas processing unit including a plurality of stacks being away from each other and in parallel, each stack including a dielectric substrate, and a first to a third electrode, the dielectric substrate having a first and a second main surface, the first and second electrodes being respectively disposed on the first and second main surfaces, the third electrode being disposed inside the dielectric substrate;
a flow forming unit that forms a flow of a target gas flowing toward the gas processing unit;
an AC power supply that applies an AC voltage across the first, second electrodes and the third electrode so as to generate plasma induced flows of the target gas between the dielectric substrates;
a first filter that is disposed at an upstream of the gas processing unit, and remove ozone; and
a second filter that is disposed at a downstream of the gas processing unit, and remove ozone.

2. The gas processing apparatus according to claim 1, wherein
the first filter contains activated carbon.

3. The gas processing apparatus according to claim 1, wherein
the second filter contains a metal catalyst.

4. The gas processing apparatus according to claim 1, wherein
the second filter is disposed by being separated from end portions on the downstream side of the third electrodes by not less than 1 mm nor more than 5 mm.

5. The gas processing apparatus according to claim 1, wherein
the second filter has a heater.

6. The gas processing apparatus according to claim 5, further comprising
a controller that controls to apply the AC voltage to the plurality of stacks when a temperature of the second filter exceeds a threshold value.

7. The gas processing apparatus according to claim 6, wherein
the threshold value is 50° C. or more.

8. The gas processing apparatus according to claim 1, further comprising
a second controller that controls so that the gas processing unit or the flow forming unit operates in a first mode, and then the gas processing unit or the flow forming unit operates in a second mode in which an operation level is higher than that of the first mode.

9. The gas processing apparatus according to claim 8, wherein
the voltage applied to the gas processing unit in the second mode is larger than that in the first mode.

10. The gas processing apparatus according to claim 8, wherein
a flow rate of the flow formed by the flow forming unit in the second mode is larger than that in the first mode.

11. The gas processing apparatus according to claim 8, wherein
the second controller controls to switch from the first mode to the second mode after the operation in the first mode continues for five minutes or more.

12. The gas processing apparatus according to claim 1, further comprising
a third controller that controls to make the flow forming unit and the gas processing unit operate sequentially.

13. The gas processing apparatus according to claim 1, further comprising
a third controller that switches a first state to a second state, in the first state the gas processing unit or the flow forming unit operating in a third mode to discharge ozone with a first concentration, in the second state the gas processing unit or the flow forming unit operating in a fourth mode to discharge ozone with a second concentration higher than the first concentration.

14. The gas processing apparatus according to claim 9, wherein
a flow rate of the flow formed by the flow forming unit in the second mode is larger than that in the first mode.
